(19) United States
(12) Patent Application Publication
(10) Pub. No.: US 2011/0097363 A1
(43) Pub. Date: Apr. 28, 2011

(54) Therapeutically Effective Preparations of Insulin

(75) Inventors: Fred Feldman, Kensington, NH (US); Zhengmao Li, Newmarket, NH (US); Jeffrey Banas, Raymond, NH (US); Carl Reppucci, North Andover, MA (US)

(73) Assignee: CPEX PHARMACEUTICALS, INC., Exeter, NH (US)

(21) Appl. No.: 12/902,397

(22) Filed: Oct. 12, 2010

Related U.S. Application Data
(60) Provisional application No. 61/253,915, filed on Oct. 22, 2009.

(51) Int. Cl. A61K 38/28; A61K 9/107; A61P 3/10

(52) U.S. Cl. 424/400; 514/5.9

(57) ABSTRACT

The present invention provides a preparation of a therapeutically effective amount of insulin with a concentration of A21 desamido insulin greater than about 2% (w/w).

### FIELD OF THE INVENTION

The present invention relates to therapeutically effective preparations of insulin with concentrations of A21 desamido insulin greater than about 2% (w/w).

### BACKGROUND OF THE INVENTION

Insulin is a hormone that induces transport of glucose from the blood to the inside of a cell, where the glucose provides a source of energy. Type I diabetes is characterized as a metabolic disorder in which the body does not produce insulin. Harrison's Principles of Internal Medicine (2005), Diabetes Mellitus, p. 2152. Type II diabetes is a group of disorders characterized by varying degrees of insulin resistance, impaired insulin secretion and increased glucose production. Harrison's at p 2152. People who suffer from Type I and Type II diabetes often require the administration of exogenous insulin to control blood-sugar. Harrison's at p. 2152.

A key factor in maintaining blood glucose control where there is limited or absent insulin production, is the timely delivery of insulin in doses that match the increase in blood glucose after a meal. If too much insulin is delivered, hypoglycemia can occur. In contrast, if too little insulin is delivered, hyperglycemia may result. If the delivery of postprandial insulin is delayed, the subject may experience hyperglycemia followed by a period of hypoglycemia. Both conditions can cause serious clinical complications.

The most common regimen of insulin treatment is subcutaneous injection of short term, fast acting insulin before meals in conjunction with administration of a longer, slower acting formulation of insulin. More recently, however, insulin has been shown to be effective when delivered by other methods, such as by nasal administration, as shown for example in U.S. Pat. No. 7,112,561, or by pulmonary administration (http://www.mannkindcorp.com/diabetes-trials.aspx, Sep. 6, 2009).

Human insulin has two peptide chains, referred to as the A chain, with 21 amino acids, ending with asparagine, and the B chain with 30 amino acids. The A and B chains are connected by two disulfide bridges, which hold the molecule together. The A chain also has a third internal disulfide bridge. Brange, et al., Stability of Insulin, Kluwer Academic Publishers, 1994.

Insulin can undergo chemical degradation by deamidation of the glutamine or asparaginine residues to form a free carboxylic acid. Id. at 327. The reaction occurs at the A21 residue and is accelerated by storage under acidic conditions. When stored at 2-8° C. neutral pH, there is little deamindation after 2 years, <7%. Id. at 341. The Food and Drug Administration (“FDA”) and the United States Pharmacopeia (“USP”) have limited the amount of A21 insulin that can be present in some pharmaceutical preparations to less than 1.5%. E.g., USP 32, Lispro May 1, 2009, p. 2645-46. We have found that preparations containing as much as 40-50% (w/w) A21 desamido insulin are equally effective in controlling blood glucose as preparations containing less than 1.5% (w/w).

### SUMMARY OF THE INVENTION

It is an object of the present invention to provide a preparation of a therapeutically effective amount of insulin with a concentration of A21 desamido insulin greater than about 2% (w/w). We have discovered that there is unexpected activity of preparations having a high A21 desamido insulin concentration. “Activity” is measured by BioIdentity and BioAssay tests, described herein, performed according to USP 121 (2009), which is incorporated by reference in its entirety herein. The preparations of the present invention contain more than about 2% (w/w) of A21 desamido insulin as compared to the “reference product,” which contains less than about 2% (w/w) of A21 desamido insulin.

These preparations containing more than about 2% (w/w) of A21 desamido insulin can be formulated for nasal administration (e.g., nasal spray), injection, transdermal delivery, oral delivery or inhalation. These preparations can be in a suspension or an emulsion. The insulin of the inventive preparation can be formulated with a permeation enhancer, which can be a Hsieh enhancer, and a liquid carrier.

In one embodiment, this invention provides a preparation where the A21 desamido insulin may range from about 2% (w/w) to about 80% (w/w). The invention further provides for preparations where the A21 desamido insulin may range from about 5% (w/w) to about 70% (w/w), 10% (w/w) to about 60% (w/w), 20% (w/w) to about 50% (w/w) or 30% (w/w) to about 40% (w/w).

The preparations may be stored for at least about 1 month, at least about 6 months, at least about 12 months, at least about 24 months or at least about 36 months at about 5° C. (refrigerated conditions) or about 25° C. (room temperature) (referred to herein as the “Conditions”). Under the foregoing Conditions, the pH of the preparation may not vary more than about 0.5 pH units. Furthermore, the therapeutic efficacy of these preparations as compared to the reference product can be confirmed by testing according to USP 121 (2009), which is incorporated by reference in its entirety herein.

In one embodiment, the osmolality of these preparations does not vary more than about 0.5% when stored under the Conditions. In another, the D₉₀ measurement of the oil droplets in the emulsion of insulin in the preparation is equal to about 0.4-5 microns when stored under the Conditions. Further, the insulin content of the inventive preparation is from about 90% to about 115% of the reference product when stored under the Conditions. “Insulin content,” as referred to herein, shall refer to the amount of deamidated insulin, with greater than about 2% (w/w) A21 desamido insulin, and native insulin, with less than about 2% (w/w) A21 desamido insulin, in the preparation as confirmed by the High Performance Liquid Chromatography (HPLC) assay described herein.

### BRIEF DESCRIPTION OF THE DRAWINGS

FIG. 1 depicts the pH of the preparation of this invention when stored over about a 24 month period under the following conditions: about 5° C. upright, about 25° C. upright and about 5° C. inverted.

FIG. 2 depicts the osmolality of the preparation of this invention when stored over about a 24 month period under the following conditions: about 5° C. upright, about 25° C. upright and about 5° C. inverted.

FIG. 3 depicts the insulin content of the preparation of this invention as compared to Nasulin® 1% (Intranasal 1% (w/w) Insulin Spray), the reference product, over about a 24 month period when stored under the following conditions: about 5° C. upright, about 25° C. upright and about 5° C. inverted.

FIG. 4 depicts the percentage of A21 of the preparation of this invention when stored over about a 24 month period under the following conditions: about 5° C. upright, about 25° C. upright and about 5° C. inverted.

FIG. 5 depicts the results of a CPE-215 (oxycylcohexadecan-2-one) assay of the content of a preparation of this invention when stored over about a 24 month period under the following conditions: about 5° C. upright, about 25° C. upright and about 5° C. inverted.

FIG. 6 depicts the emulsion size (by diameter in μm) of a preparation of this invention over about a 24 month period when stored under the following conditions: about 5° C. upright, about 25° C. upright and about 5° C. inverted.

FIG. 7 depicts the results of a test of the preparation of this invention complying with the Guidance for Industry for Nasal Spray and Inhalation Solutions, Suspensions, and Spray Drug Products when stored over about a 24 month period.

FIG. 8 depicts the results of a USP 121 BioIdentity test of a preparation of this invention over about a 24 month period when stored under the following conditions: about 5° C. upright, about 25° C. upright and about 5° C. inverted.

FIG. 9 depicts the results of the USP 121 rabbit blood-sugar quantitative test of a preparation of this invention stored over about an 18 month period under at about 5° C., about 25° C. and about 40° C.

### DETAILED DESCRIPTION OF THE INVENTION

The composition of the present invention comprises a therapeutically effective amount of insulin wherein the insulin has greater than about 2% (w/w) of the A21 desamido insulin degradation product present. The term “therapeutically effective amount of insulin” as used herein means, an amount of insulin in the preparation of the invention sufficient to achieve a clinically significant control of blood glucose concentrations in a human diabetic patient with either Type I or Type II diabetes.

Insulin

Any type of insulin may be used with the methods and formulations of the present invention, including, without limitation, native insulin, i.e., purified from bovine or porcine sources, recombinant human insulin, proinsulin, semi-synthetic human insulin, zinc insulin, protamine zinc insulin, isophane insulin, radio-iodinated insulin, any insulin analogues, derivatives, polymorphs, metabolites, pro-drugs, salts, and/or hydrates. Examples of insulin analogues are human insulin, insulin lispro, insulin aspart, insulin glulisine, insulin glargine, and insulin detemir. Zinc salts of insulin may also be used. Derivatives of insulin include, insulin that has been modified at the internal or terminal amino acids, for example, lysine/proline-substituted insulin derivatives. Rapid, intermediate- and long-acting insulins may also be used with the methods and systems of the present invention. Bethel et al. Journal of the American Board of Family Practice, 18:199-204 (2005). Short-acting and pre-mixed insulins may also be used.

Dosing regimens of insulin will vary among the various types of insulins as well as among patients. Dosages of insulin will be determined by the healthcare provider who is familiar with the patient's metabolic needs, eating habits, and other lifestyle variables. Physicians' Desk Reference, pp. 1834, 1844-46 (2009). The doses may be delivered in single or multiple doses and may contain equal or different amounts of insulin depending on the patient's needs.

A21 Desamido Insulin

Human insulin has two peptide chains, referred to as the A chain, with 21 amino acids, ending with asparagine, and the B chain with 30 amino acids. The A and B chains are connected by two disulfide bridges, which hold the molecule together. The A chain also has a third internal disulfide bridge. Brange, et al., Stability of Insulin, Kluwer Academic Publishers, 1994.

The structural integrity of insulin can become compromised during storage. Temperature, pH, humidity and time can effect the integrity of insulin. Chemical instability refers to covalent modification of the primary structure leading to bond formation or cleavage. Such covalent changes are irreversible and may lead to formation of less bioactive molecules. The stability of insulin is mainly compromised by the formation of soluble aggregates (dimers and polymers) over time, even though insulin is usually stored at temperatures of no more than about 5° C. The shelf-life of insulin can also be compromised through the formation of insoluble aggregates (fibrils) as a result of being shaken, for example when being transported. U.S. Pat. No. 6,174,856. Chemical deterioration can be observed when aged samples are analyzed by disc electrophoresis, size exclusion chromatography or High Performance Liquid Chromatography (HPLC). Brange, et al., Stability of Insulin, Kluwer Academic Publishers, 1994.

Chemical deterioration of insulin during storage most often occurs through deamidation, which is a reaction that takes place when the side chain amide group in glutamine (“Gln”) or asparagine (“Asn”) residues are hydrolyzed to form a free carboxylic acid. Insulin contains six residues where deamidation can occur, one of which is Asn²¹ or A21 residue. Brange, et al., Stability of Insulin, Kluwer Academic Publishers, 1994.

A21 desamido insulin is the major product formed during mild acid hydrolysis of insulin. Hoppe Seylers Z., Physiol. Chem. 1978 September; 359(9):1229-36. Laboratory studies of human insulin in solutions between pH 2-5 and 35° C. show that both deamidation and covalent dimerization at the C-terminal of Asn²¹ occur by way of a nucleophilic attack on the protonated C-terminal carboxylic acid onto the side-chain amide, which forms a reactive cyclic anhydride intermediate. The intermediate quickly reacts with water to form A21 desamido insulin. Stickley, et. al., Solid-State Stability of Human Insulin I. Mechanism and the Effect of Water on the Kinetics of Degradation in Lyophiles from pH 2-5 Solutions, Pharm. Res., Vol. 13, No. 8 (1996).

Until now, it was thought that insulin with concentrations of A21 desamido insulin over about 1.50% (w/w) had less activity than those with lower concentrations of A21 desamido insulin. USP 32, May 1, 2009. Regulatory agencies, such as the FDA mandate that levels of A21 desamido insulin be less than about 2% (w/w) of certain insulin formulations. We have unexpectedly discovered that concentrations of A21 desamido insulin over about 2% (w/w) are as effective in controlling blood glucose concentrations in patients as insulin formulations with less than about 2% (w/w) A21 desamido insulin.

In preparations encompassed by the present invention, A21 desamido insulin may be present in concentrations ranging from greater than about 2% (w/w), about 2% (w/w) to about 80% (w/w), about 5% (w/w) to about 70% (w/w), about 10% (w/w) to about 60% (w/w), about 20% (w/w) to about 50% (w/w) or about 30% (w/w) to about 40% (w/w). Concentrations greater than about 80% (w/w) are also encompassed by the present invention. The insulin preparation containing A21 desamido insulin may be stored at room temperature, about 25° C., at about 5° C. or at about 10° C. for periods of time ranging from about 1, 2, 3, 4, 5, 6, 7, 8, 9, 10, 11, 12, 24 or 36 months.

Types of Insulin Preparations

The invention encompasses insulin with the aforementioned concentrations of A21 desamido insulin alone or in formulated preparations. The present invention can be delivered by inhalation, nasally, intravenously, orally, subcutaneously, intramuscularly or transdermally. The present pharmaceutical preparation may be administered by any method known in the art, including, without limitation, oral, nasal, subcutaneous, intramuscular, intravenous, transdermal, rectal, sub-lingual, mucosal, ophthalmic, spinal, intrathecal, intra-articular, intra-arterial, sub-arachnoid, bronchial and lymphatic administration, and other dosage forms for systemic delivery of active ingredients. The composition may also be delivered topically.

The present invention can be formulated into a suspension that yields a heterogeneous fluid containing solid particles that are sufficiently large for sedimentation, comprising the active ingredient and a vehicle. The active ingredient is pharmaceutically active, e.g. insulin, and is insoluble in the vehicle in that the insulin is not dissolved and is suspendable. Suspensions are defined as a class of materials in which one phase, as solid, is dispersed in a second phase, generally a liquid. The vehicle may be a pharmaceutically acceptable, aqueous, and suspension-stabilizing, comprising a thickener component and a carrier component, and may include organoleptic components. The present invention can also be an emulsion comprising a mixture of two unblendable liquids, one of which is insulin, in which one liquid is dispersed in the other.

The preparations of the invention are generally employed in a dosing regimen that is dependent on the patient being treated. Thus, the frequency of the use and the amount of the dose may vary from patient to patient. As known in the art, the treatment of a disease such as diabetes through insulin therapy varies from patient to patient, and based on known insulin therapy and the teachings herein, one skilled in the art can select the dosing regimen and dosage for a particular patient or patients. The treatment regimen may require administration over extended periods of time, for example, for several weeks or months; the treatment regimen may require administration over years.

Insulin is commonly delivered by a parenteral route of administration through intravenous, intramuscular or subcutaneous injection. For parenteral formulations, the carrier will usually comprise sterile water, although other ingredients, for example, ingredients that aid solubility or for preservation, may be included. Injectable solutions may also be prepared in which case appropriate stabilizing agents may be employed. Parenteral administration may comprise any suitable form of systemic delivery or delivery directly to the central nervous system. Administration may be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

The inventive preparation can be delivered in an oral dosage form; any of the usual pharmaceutical media may be employed with such oral dosage forms. Thus, for liquid oral preparations, such as, for example, suspensions, elixirs and solutions, suitable carriers and additives include water, glycols, oils, alcohols, flavoring agents, preservatives, coloring agents and the like. For solid oral preparations such as, for example, powders, capsules and tablets, suitable carriers and additives include starches, sugars, diluents, granulating agents, lubricants, binders, disintegrating agents and the like.

In one embodiment of the inventive preparation, insulin in a dry powder form can be inhaled into the lungs. www.mannkindcorp.com. Typical delivery systems for inhalable agents include nebulizer inhalers, dry powder inhalers, and metered-dose inhalers.

Formulations for rectal or vaginal administration may be presented as a suppository with a suitable carrier such as cocoa butter, hydrogenated fats, or hydrogenated fatty carboxylic acids.

Transdermal formulations may be prepared by incorporating the insulin in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer. If the composition is in the form of a gel, the composition may be rubbed onto a membrane of the patient, for example, the skin, preferably intact, clean, and dry skin, of the shoulder or upper arm and or the upper torso, and maintained thereon for a period of time sufficient for delivery of androgen to the blood serum of the patient. The composition of the present invention in gel form may be contained in a tube, a sachet, or a metered pump. Such a tube or sachet may contain one unit dose of the composition. A metered pump may be capable of dispensing one metered dose of the composition.

Ointment and creams may, for example, be formulated with an aqueous or oily base with the addition of suitable thickening and/or gelling agents. Lotions may be formulated with an aqueous or oily base and, in general, also include one or more of the following: stabilizing agents, emulsifying agents, dispersing agents, suspending agents, thickening agents, coloring agents, perfumes, and the like.

The present invention may be delivered as a nasal spray. In such an embodiment, the liquid carrier may be water with the insulin being dispersed or dissolved in the water in a therapeutically effective amount. Nasal and other mucosal spray formulations (e.g. inhalable forms) can comprise purified aqueous solutions of the active compounds with or without preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like. The pharmaceutical formulations of the present invention may comprise a pharmaceutically effective amount of insulin and a permeation enhancer. U.S. Pat. Nos. 7,112,561, 7,244,703 and 7,320,968, which are incorporated by reference in their entirety herein. The permeation enhancer may be a Hsieh enhancer having the following structure:

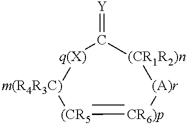

wherein X and Y are oxygen, sulfur or an imino group of the structure

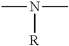

or ═N—R with the proviso that when Y is the imino group, X is an imimo group, and when Y is sulfur, X is sulfur or an imino group, A is a group having the structure

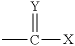

wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11, and said compound will enhance the rate of the passage of the drug across body membranes. Hereinafter these compounds are referred to as enhancers. When R, R₁, R₂, R₃, R₄, R₅ and R₆ is alkyl, it may be methyl, ethyl, propyl, isopropyl, butyl, isobutyl, sec-butyl, amyl, hexyl, and the like. Such permeation enhancers are described in U.S. Pat. No. 5,023,252 and U.S. Pat. No. 5,731,303.

Examples of the permeation enhancers are the cyclic lactones (the compounds wherein both X and Y are oxygen, (q is 1 and r is 0), the cyclic diesters (the compounds wherein both X and Y are oxygen, and both q and r are 1), and the cyclic ketones (the compounds wherein both q and r are 0 and Y is oxygen). In the cyclic diesters m+n is preferably at least 3. In the cyclic ketones m+n is preferably from 11 to 15 and p is preferably 0. Examples of the enhancers for use in the present invention are macrocyclic enhancers. The term “macrocyclic” is used herein to refer to cyclic compounds having at least 12 carbons in the ring, including: (A) macrocyclic ketones, for example, 3 methylcyclopentadecanone (muscone), 9-cycloheptadecen-1-one (civetone), cyclohexadecanone, and cyclopentadecanone (normuscone); and (B) macrocyclic esters, for example, pentadecalactones such as oxacyclohexadecan-2-one (cyclopentadecanolide, ω-pentadecalactone). Other permeation enhancers that may be used are the simple long chain esters that are Generally Recognized As Safe (GRAS) in the various pharmacopoeial compendia (see, also, www.fda.gov, e.g., Guidance for Industry, Nonclinical Studies for the Safety Evaluation of Pharmaceutical Excipients). These may include simple aliphatic, unsaturated or saturated (but preferably fully saturated) esters, which contain up to medium length chains. Non-limiting examples of such esters include isopropyl myristate, isopropyl palmitate, myristyl myristate, octyl palmitate, and the like. The enhancers are of a type that are suitable for use in a pharmaceutical composition. An artisan of ordinary skill will also appreciate that those materials that are incompatible with or irritating to mucous membranes should be avoided.

The enhancer is present in the composition at a concentration effective to enhance penetration of the insulin across the nasal mucosa. Various considerations should be taken into account in determining the amount of enhancer to be used. Such considerations include, for example, the amount of flux (rate of passage through the membrane) achieved and the stability and compatibility of the components in the formulations. The enhancer is generally used in an amount of about 0.01 to about 25% (w/w) the composition, more generally in an amount of about 0.1 to about 15% (w/w) the composition, and in some embodiments in an amount of about 0.5 to about 15% (w/w) the composition. U.S. Pat. No. 7,112,561.

The pharmaceutical formulations of the present invention may comprise a therapeutically effective amount of insulin, a permeation enhancer, and a liquid carrier. The present formulations may be at an acidic pH. The liquid carrier is present in the composition in a concentration effective to serve as a suitable vehicle for the compositions of the present invention. In general, the carrier is used in an amount of about 40 to about 98% (w/w) of the composition and in some embodiments in an amount of about 50 to about 98% (w/w) of the composition.

In one embodiment of the present invention, the nasal administration of insulin is through a nasal spray which uses water as the liquid carrier with insulin being dispersed or dissolved in the water in a therapeutically effective amount. In another embodiment, the permeation enhancer is emulsified in the aqueous phase that contains the insulin. The emulsification may be effected through the use of one or more suitable surfactants. Any suitable surfactant or mixture of surfactants can be used in the practice of the present invention, including, for example, anionic, cationic, and non-ionic surfactants. Examples of non-ionic surfactants are PEG-60 corn glycerides, PEG-20 sorbitan monostearate, phenoxy-poly(ethyleneoxy)ethanol, sorbitan monooleate, and the like. In general the surfactant is present in an amount less than about 2% (w/w) the composition. In another embodiment, the surfactant may be present in amounts less than about 1.5% (w/w), less than about 1.3% (w/w), less than about 1% (w/w), or less than about 0.3% (w/w).

The inventive preparation can have immediate release, sustained release, delayed-onset release or any other release profile known to one skilled in the art. The composition of the present invention may be formulated by the use of conventional means, for example, by mixing and stirring the ingredients. Conventional equipment may be used.

### SPECIFIC EXAMPLES

The following Examples illustrate various embodiments of the invention and are not to be regarded as limiting.

### Example 1

### Stability/Efficacy Tests

The following tests were all performed using Nasulin® 1% (Intranasal 1% (w/w) Insulin Spray), the reference product, for the treatment for patients with Type I and Type II diabetes under U.S. IND No. 68,464. Stability programs were developed with guidance from the Division of Endocrinologic and Metabolic Drug Products. Clinical supplies were manufactured and tested at Catalent Pharma Solutions (formerly Cardinal Health) at Research Triangle Park, North Carolina. Three clinical lots of finished drug product were manufactured at that site and tested and are designated by lot numbers Lot 1, Lot 2 and Lot 3. Release and stability testing of finished drug product lots were performed according to qualified test methods shown in Table 1 with the exception of the insulin assay (BioIdentity test), which was tested according to USP 121, which is incorporated herein by reference in its entirety. Specifications for release and stability are shown in Table 2. The stability testing schedules for clinical lots shown in Tables 3 and 4 include testing vials from each lot at multiple temperatures: refrigerated temperature (about 2-8° C., ambient humidity), room temperature (about 25° C., 60% relative humidity), and elevated temperature conditions (about 40° C., 75% relative humidity). Stability vials for the study were stored in controlled temperature chambers in two orientations: upright and inverted as indicated.

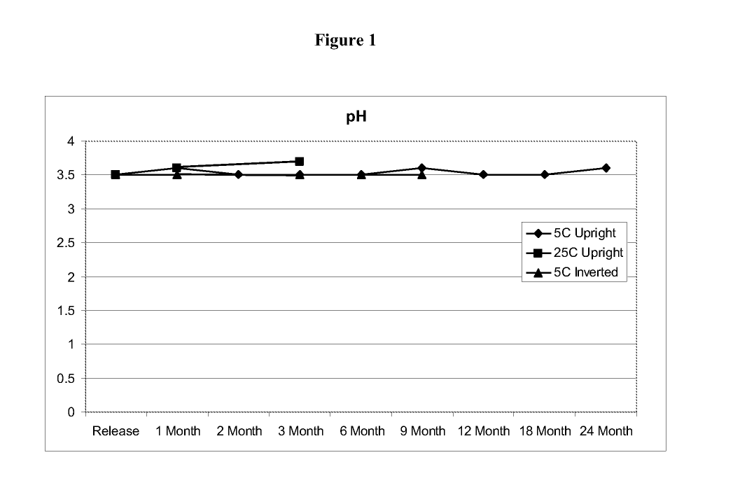

A number of tests including, pH, osmolality, insulin assay, related substances, CPE-215 assay (oxycylohexadecan-2-one), emulsion size, spray association content, shot weight, net content, BioIdentity and BioAssay were performed on three different lots of intranasal insulin, Nasulin®, to determine the stability and efficacy of the inventive preparation. FIGS. 1-9 show graphic trend analyses over time for the results of Lot 3 for each of these tests.

Test A. pH

FIG. 1 depicts the pH of insulin over about a 24 month period at temperatures of about 5° C. and about 25° C. in the upright position and about 5° C. in the inverted position. The test of the insulin was performed using USP 791, which is incorporated herein by reference in its entirety. As shown in FIG. 1, the pH of insulin was stable within about 3.5±0.5 for all monitored conditions throughout the 24 month period.

Test B. Osmolality

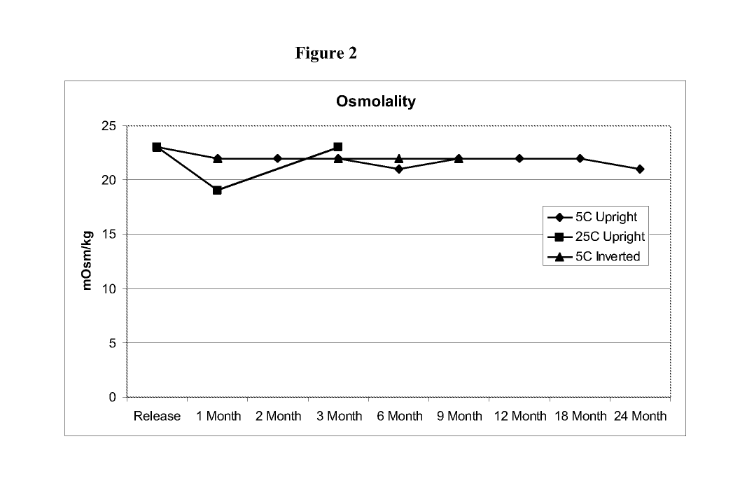

FIG. 2 depicts the osmolality of insulin over about a 24 month period at temperatures of about 5° C. and about 25° C. in the upright position and about 5° C. in the inverted position. The test of the insulin was performed using USP 785, which is incorporated herein by reference in its entirety. The results show that the osmolality of insulin was consistently below the specification of ≦100 mOsm/kg for all monitored conditions and did not vary by more than 0.5%.

Test C. Insulin Assay

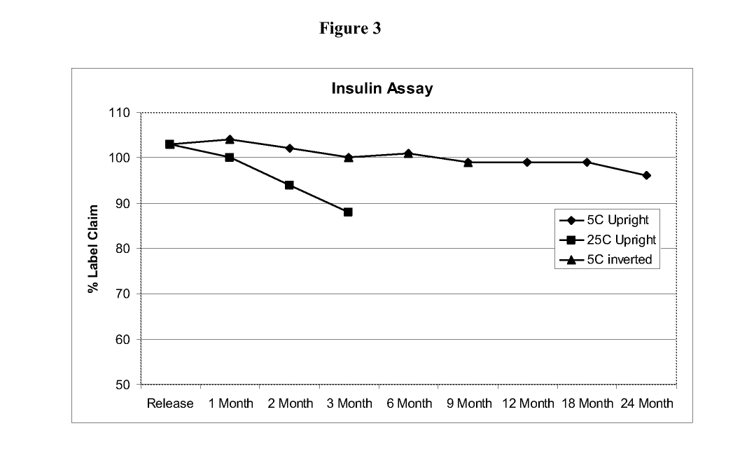

FIG. 3 depicts an insulin assay in which samples of insulin were measured by High Performance Liquid Chromatography (HPLC) over about a 24 month period at temperatures of about 5° C. and about 25° C. in the upright position and at about 5° C. in the inverted position. The results show the sum of the areas of native insulin and A21 desamido insulin. The results show that the samples of insulin were consistently within the specification of about 90% to about 115% of the reference product stored and refrigerated at about 5° C., even after about 24 months at which point the sample contained greater than about 30% A21 desamido insulin. The samples were also within the specification of about 90% to about 115% of the reference product for at least about two months for the lots stored at room temperature, at which point the sample contained greater than about 50% A21 desamido insulin.

Test D. Related Substances

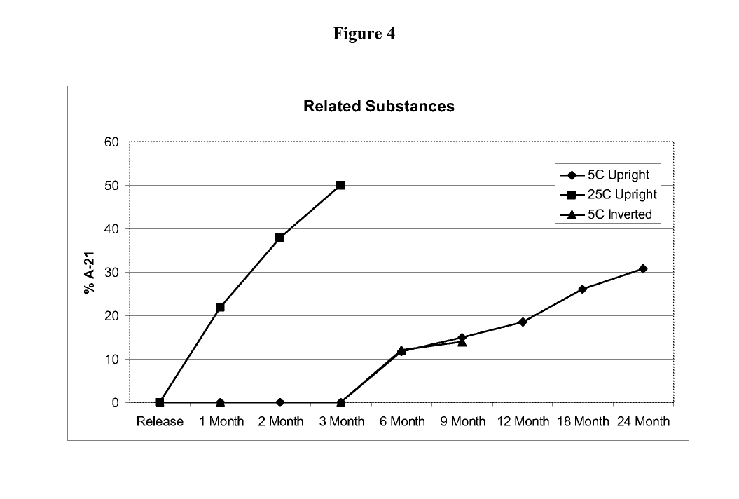

FIG. 4 depicts the percentage of A21 desamido insulin in the insulin samples, as measured by High Performance Liquid Chromatography (HPLC), over about a 24 month period at temperatures of about 5° C. and about 25° C. in the upright position and at about 5° C. in the inverted position. No related substances other than A21 desamido insulin were observed above the limit of quantitation of about 0.0012%. When stored at refrigerated conditions, A21 desamido insulin increased to over about 20% after about twelve months. When stored at room temperature, about 25° C., A21 desamido insulin increased to over about 20% after about one month and to over about 80% after about six months. When stored at about 40° C., A21 desamido insulin increased to about 90% after about one month.

Test E. CPE-215 Assay (Oxycylcohexadecan-2-One)

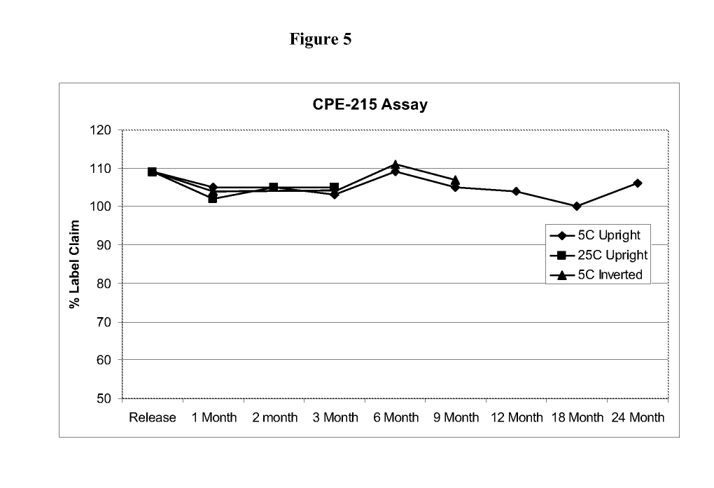

This assay, the results of which are set forth in FIG. 5, was performed to determined the levels of the macrocyclic enhancer, oxacyclohexadecan-2-one, in the formulations. The formulations were assayed by a gas chromatography method through direct dilution of the target formulation. FIG. 5 shows that the samples were consistently within the specification of about 80% to about 120% of the reference product at all storage conditions and times tested.

Test F. Emulsion Size

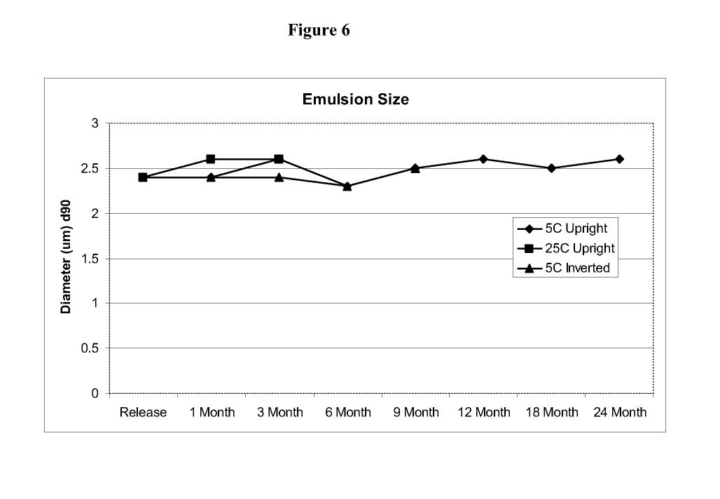

This study analyzed the particle size distribution by laser diffraction of the bulk insulin. The results, as shown in FIG. 6, show that the oil droplets in the emulsion of insulin passed the specification of D₉₀ equal to about 0.4-5 microns at all storage conditions and times tested over the 24 month period. D₉₀ represents 90th percentile of the particle size distribution as measured by volume.

Test G. Spray Association Content

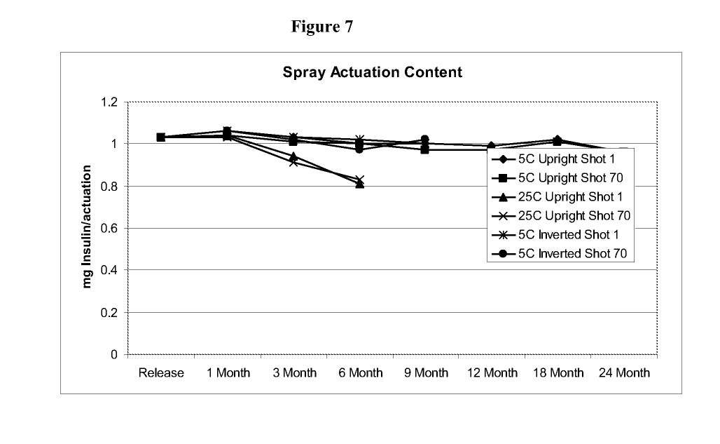

As shown in FIG. 7, when insulin was stored at about 5° C. for about 24 to about 36 months or stored at about 25° C. for at least one month: 1) the active was not outside of about 80% to about 120% of the reference product for more than 2 of 20 determinations from 10 container; 2) none of the determinations was outside of about 75% to about 125% of the reference product; and 3) the mean for each of the beginning and end determinations was not outside of about 85% to about 115% of the reference product.

Test H. BioIdentity

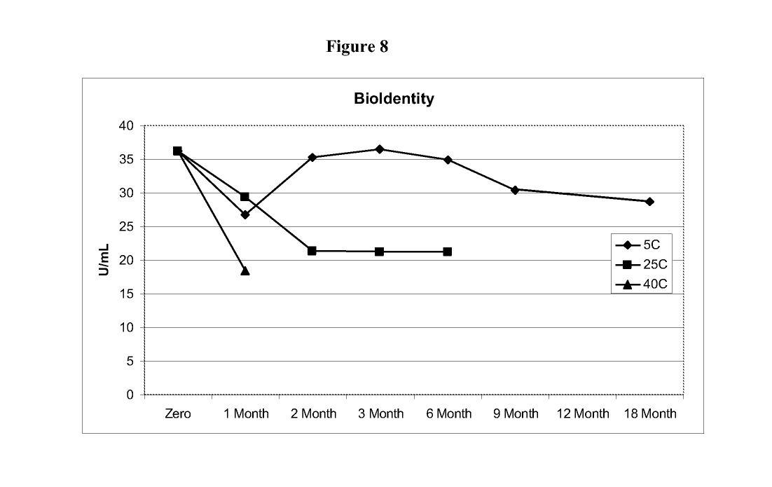

This qualitative insulin assay was performed according to USP 121, which is incorporated by reference in its entirety. Using the USP 121 procedure, this test compared the blood-sugar values of 8 rabbits injected with insulin. A sample is said to “pass” this test when the insulin activity is at least 15 USP Units/mg for insulin. FIG. 8 shows that the insulin samples with greater than 2% (w/w) A21 desamido insulin continued to pass the USP 121 insulin assay when stored at refrigerated conditions even after about 24 to about 36 months, at which point the sample contained greater than about 20% (w/w) A21 desamido insulin, and independent of the level of A21 desamido insulin. The samples also passed the USP 121 insulin assay when stored at room temperature for at least about six months, at which point the sample contained greater than about 80% (w/w) A21 desamido insulin, and independent of the level of A21 desamido insulin. The samples continued to pass the USP 121 insulin assay when stored after about one month at about 40° C., at which point the sample contained greater than about 90% (w/w) A21 desamido insulin.

Test K. BioAssay

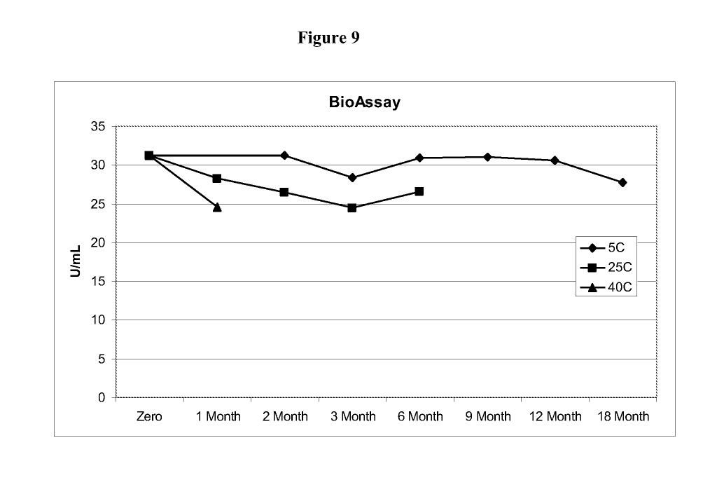

This quantitative insulin assay, the results of which are set forth in FIG. 9, was performed according to USP 121 and compared the blood-sugar values of a number of rabbits sufficient to reach a 95% confidence interval, that were injected with insulin. The results of this test show that the insulin activity was about 25 to about 30 U/mL for all temperatures throughout the 18 month period.

### Example 2

### Blood Glucose Levels

On test day 1, twelve human patients with Type II diabetes will be treated with the preparation of the insulin having greater than about 2% (w/w) of the A21 desamido insulin invention (“High Concentration A21 Insulin”). The percentage of A21 may range from about 2-80% (w/w), 5-70% (w/w), 10-60% (w/w), 20-50% (w/w) or about 30-40% (w/w). Prior to administration of the High Concentration A21 Insulin, blood will be drawn from each of the patients to determine baseline blood glucose levels for each patient. After administration of High Concentration A21 Insulin, blood will be drawn at regular time points throughout a 24 hour period to determine blood glucose levels at each time point.

On test day 2, which may or may not be a day consecutive to test day 1, the same twelve human patients with Type II diabetes will be treated with the preparation of the insulin having less than about 2% (w/w) of the A21 desamido insulin invention (“Low Concentration A21 Insulin”). Prior to administration of the Low Concentration A21 Insulin, blood will be drawn from each of the patients to determine baseline blood glucose levels for each patient. After administration of Low Concentration A21 Insulin, blood will be drawn at regular time points throughout a 24 hour period to determine blood glucose levels at each time point.

These two studies will be compared to investigate whether the activity of High Concentration A21 Insulin is significantly less than the Low Concentration A21 Insulin.

### Example 3

### HbA_{1c} Test

Glycosylated hemoglobin is a type of glycosylated protein that forms when excessive levels of sugar in the blood combine with proteins. Glycosylated proteins such as glycosylated hemoglobin are substantially insoluble and give rise to thickening of the walls of veins and arteries, and thickening of the myelination of nerves. Glycosylated hemoglobin is composed of three components; one of which is HbA_{1c}. HbA_{1c} levels are commonly measured to determine effectiveness of diabetes treatment. Low levels of HbA_{1c} in a diabetic patient's blood, usually between about 6 and 7%, are a good indication that the treatment regime is effective and the risk of secondary problems related to glycosylated hemoglobin is low. U.S. Pat. No. 6,421,633.

On test day 1, blood will be drawn from twelve human patients with Type II diabetes to determine baseline amounts of HbA_{1c}. These twelve patients will then be treated, for about 6 to 8 weeks, with the preparation of the insulin having greater than about 2% (w/w) of the A21 desamido insulin invention (“High Concentration A21 Insulin”). The percentage of A21 may range from about 2-80% (w/w), 5-70% (w/w), 10-60% (w/w), 20-50% (w/w) or about 30-40% (w/w). On test day 2, which will occur after about 6 to 8 weeks of treatment with the High Concentration A21 Insulin, blood will be drawn from each of the twelve patients.

Levels of HbA_{1c} from test days 1 and 2 will be compared in each of the twelve patients to determine whether levels of HbA_{1c} are about 6 to 7% or less, indicating that treatment with the High Concentration A21 Insulin is effective.

TABLE 1

| Test (number of replicates) | ATM Number |
|---|---|
| Appearance (n = 3) | ATM-MAG-M0003 |
| pH (n = 3) | cUSP <791> |
| Osmolality (n = 3) | cUSP <785> |
| Insulin Assay and Related Substances (n = 3) | ATM-BFO-M0003 |
| CPE-215 Assay (n = 3) | ATM-BFO-M0002 |
| Emulsion Size (n = 3) | ATM-BFO-M0001 |
| Spray Actuation Content (n = 30 bottles, | ATM-BFO-M0005 |
| Tier 1 - 10 bottles; Tier 2 - 20 additional bottles) | |
| Priming (Individual shot weights for 5 actuations | ATM-BFO-M0005 |
| from 10 bottles) | |
| Droplet Size Distribution by Laser Diffraction | ATM-BFO-M0004 |
| (n = 3 bottles, 1 spray each at 2 distances) | |
| Drug Content (Insulin) in Small Droplets by | ATM-BFO-M0007 |
| Cascade Impaction (n = 3 bottles) | |
| CPE-215 Content in Small Droplets by Cascade | ATM-BFO-M0008 |
| Impaction (n = 3 bottles) | |
| Shot Weight (n = 9 bottles, Tier 1 - 3 bottles, | ATM-BFO-M0005 |
| 10 shots per bottle through bottle life. Tier 2 - | |
| 6 additional bottles, 10 shots per bottle through | |
| bottle life) | |
| Net Content (n = 3 bottles) | ATM-BFO-M0003 |
| Insulin BioIdentity (n = 3) | NA¹ |
| Foreign Particulate Matter (n = 3) | ATM-BFO-M0006 |
| Weight Loss (n = 10 bottles) | NA² |
| Microbial Limits (n = 1) | cUSP <61> |
| Bacterial Endotoxins Test (n = 1) | cUSP <85> and |
| | ATM-MBU-M0011 |

¹NA = Not Applicable. Samples will be shipped to Pacific Biolabs for BioIdentity analysis of Insulin
²NA = Not Applicable. Bottle weights will be tracked for 10 intact bottles throughout the stability storage.

TABLE 2
Specifications

| Test | Specification |
|---|---|
| Appearance | Milky White |
| pH | 3.4 ± 0.5 |
| Osmolality | ≦100 mOsm/kg |
| Insulin Assay and Related Substances | Insulin: 90 to 115% of Label Claim |
| | Related Substances: Report % AUC of A-21 |
| | and any other impurity |
| | peak ≧ LOQ |
| CPE-215 Assay | 80 to 120% of Label Claim |
| Emulsion Size | D₉₀ should be 0.4 to 5 μm |
| Spray Actuation Content | Compliance with Guidance for Industry for Nasal |
| | Spray and Inhalation Solutions, Suspension, and |
| | Spray Drug Products.. CMC; July 2002 |
| | Tier 1: as per Guidance |
| | (1) Amount of active ingredient per |
| | spray is not outside of 80-120% of label |
| | claim for more than 2 of 20 determinations |
| | from 10 containers |
| | (2) None of the determinations is |
| | outside of 75-125% of label claim |
| | (3) Mean for each of the beginning and |
| | end determinations are not outside of |
| | 85-115% of label claim |
| | Tier 2: as per Guidance |
| Droplet Size Distribution by Laser Diffraction | Report Results |
| Priming | Report Results |
| Drug Content (Insulin) in Small | Report Results |
| Droplets by Cascade Impaction | |
| CPE-215 Content in Small Droplets | Report Results |
| by Cascade Impaction | |
| Shot Weight | 85-115 mg |
| BioIdentity | Pass |
| Net Content | Report Results |
| Foreign Particulate Matter | Report Number of Particles observed in each |
| | size range listed below: |
| | <10 μm |
| | 10 to 25 μm |
| | >25 μm |
| Weight Loss | Report Results |
| Microbial Limits | Absence of E. coli, P. aeruginosa, |
| | S. aureus and Salmonella sp |
| | NMT 10 CFU/mL for Total aerobic |
| | count and total yeast and mold |
| Endotoxins | NMT 100 EU/mL |

TABLE 3
Stability Testing Schedule for Bottles Stored Upright

| | Number of Months | | | | | | | | | |
|---|---|---|---|---|---|---|---|---|---|---|
| Condition | | 2 | | | | | | | | |
| (° C./% RH) | Initial | wks | 1 | 2 | 3 | 6 | 9 | 12 | 18 | 24 |
| Initial | A, C, F | — | — | — | — | — | — | — | — | — |
| 5° C./Amb | — | — | A | B | A | A | A | A, C | A | A, C |
| 25° C./60% RH | — | — | A, F | B | A, F | A, C, F | — | (F) | — | (F) |
| 40° C./75% RH | — | B | B | — | — | — | — | — | — | — |

A = Appearance, pH, Osmolality, Insulin Assay/Related Subs, CPE-215 Content, Emulsion Size, Spray Actuation Content, Priming, Droplet Size by Laser Diffraction, Drug Content (Insulin) in Small Droplets by Cascade Impaction, CPE-215 Content in Small Droplets by Cascade Impaction, Shot Weight, Net Content, BioIdentity, Microbial Limits and Endotoxins
B = Insulin Assay/Related Subs and CPE-215 Content
C = Foreign Particulate Matter
F = Weight Loss
( ) = Indicates optional testing

TABLE 4
Stability Testing Schedule for Inverted Bottles

| | Number of Months | | | | | | | | | |
|---|---|---|---|---|---|---|---|---|---|---|
| Condition | | 2 | | | | | | | | |
| (° C./% RH) | Initial | wks | 1 | 2 | 3 | 6 | 9 | 12 | 18 | 24 |
| Initial | — | — | — | — | — | — | — | — | — | — |
| 5° C./Amb | — | — | D | — | D | D | D¹ | — | — | — |
| 25° C./60% RH | — | — | — | — | — | D | — | — | — | — |

D = Appearance, pH, Osmolality, Insulin Assay/Related Subs, CPE-215 Content, Emulsion Size, Spray Actuation Content, Droplet Size by Laser Diffraction, Drug Content (Insulin) in Small Droplets by Cascade Impaction, CPE-215 Content in Small Droplets by Cascade Impaction, Shot Weight, Net Content, Microbial Limits and Endotoxins
¹The 9 Month pull may be delayed to 12 months. This decision will be made after a review of 6 month data.

The scope of the present invention is not limited by what has been specifically shown and described hereinabove. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

What is claimed is:

1. A preparation comprising a therapeutically effective amount of insulin wherein the insulin has greater than about 2% (w/w) of the A21 desamido insulin.

2. The preparation of claim 1 wherein the insulin is formulated for nasal administration.

3. The preparation of claim 2 wherein the insulin is formulated in a nasal spray.

4. The preparation of claim 3 wherein the insulin is formulated with a permeation enhancer and a liquid carrier.

5. The preparation of claim 4 wherein the permeation enhancer is a Hsieh enhancer.

6. The preparation of claim 1 wherein the insulin is formulated for injection.

7. The preparation of claim 1 wherein the insulin is formulated for transdermal delivery.

8. The preparation of claim 1 wherein the insulin is formulated for oral delivery.

9. The preparation of claim 1 wherein the insulin is formulated for delivery by inhalation.

10. The preparation of claim 1 wherein the insulin is a suspension.

11. The preparation of claim 1 wherein the insulin is an emulsion.

12. The preparation of claim 1 wherein the A21 desamido insulin ranges from about 2% (w/w) to about 80% (w/w).

13. The preparation of claim 12 wherein the A21 desamido insulin ranges from about 5% (w/w) to about 70% (w/w).

14. The preparation of claim 13 wherein the A21 desamido insulin ranges from about 10% (w/w) to about 60% (w/w).

15. The preparation of claim 14 wherein the A21 desamido insulin ranges from about 20% (w/w) to about 50% (w/w).

16. The preparation of claim 15 wherein the A21 desamido insulin ranges from about 30% (w/w) to about 40% (w/w).

17. The preparation of claim 1 wherein the preparation has been stored at about 5° C. for at least about 1 month.

18. The preparation of claim 17 wherein the preparation has been stored at about 5° C. for at least about 6 months.

19. The preparation of claim 18 wherein the preparation has been stored at about 5° C. for at least about 12 months.

20. The preparation of claim 19 wherein the preparation has been stored at about 5° C. for at least about 24 months.

21. The preparation of claim 20 wherein the preparation has been stored at about 5° C. for at least about 36 months.

22. The preparation of claim 1 wherein the preparation has been stored at about 25° C. for at least about 1 month.

23. The preparation of claim 22 wherein the preparation has been stored at about 25° C. for at least about 2 months.

24. The preparation of claim 23 wherein the preparation has been stored at about 25° C. for at least about 6 months.

25. The preparation of claim 24 wherein the preparation has been stored at about 25° C. for at least about 12 months.

26. The preparation of claim 25 wherein the preparation has been stored at about 25° C. for at least about 24 months.

27. The preparation of claim 26 wherein the preparation has been stored at about 25° C. for at least about 36 months.

28. The preparation of claim 1 wherein the pH of a formulation comprising the insulin does not vary more than about 0.5 pH units after at least about 1 month of storage at about 5° C.

29. The preparation of claim 28 wherein the pH of a formulation comprising the insulin does not vary more than about 0.5 pH units after at least about 6 months of storage at about 5° C.

30. The preparation of claim 29 wherein the pH of a formulation comprising the insulin does not vary more than about 0.5 pH units after at least about 12 months of storage at about 5° C.

31. The preparation of claim 30 wherein the pH of a formulation comprising the insulin does not vary more than about 0.5 pH units after at least about 24 months of storage at about 5° C.

32. The preparation of claim 31 wherein the pH of a formulation comprising the insulin does not vary more than about 0.5 pH units after at least about 36 months of storage at 5° C.

33. The preparation of claim 1 wherein the pH of a formulation comprising the insulin does not vary more than about 0.5 pH units after at least about 6 months of storage at about 25° C.

34. The preparation of claim 33 wherein the pH of a formulation comprising the insulin does not vary more than about 0.5 pH units after at least about 12 months of storage at about 25° C.

35. The preparation of claim 34 wherein the pH of a formulation comprising the insulin does not vary more than about 0.5 pH units after at least about 24 months of storage at about 25° C.

36. The preparation of claim 35 wherein the pH of a formulation comprising the insulin does not vary more than about 0.5 pH units after at least about 36 months of storage at about 25° C.

37. A preparation of claim 1 wherein therapeutic efficacy is confirmed by testing according to USP 121.

38. The preparation of claim 1 wherein the osmolality of a formulation comprising insulin does not vary more than about 0.5% after at least about 1 month of storage at about 5° C.

39. The preparation of claim 38 wherein the osmolality of a formulation comprising insulin does not vary more than about 0.5% after at least about 6 months of storage at about 5° C.

40. The preparation of claim 39 wherein the osmolality of a formulation comprising insulin does not vary more than about 0.5% after at least about 12 months of storage at about 5° C.

41. The preparation of claim 40 wherein the osmolality of a formulation comprising insulin does not vary more than about 0.5% after at least about 24 months of storage at about 5° C.

42. The preparation of claim 41 wherein the osmolality of a formulation comprising insulin does not vary more than about 0.5% after at least about 36 months of storage at about 5° C.

43. The preparation of claim 1 wherein the osmolality of a formulation comprising insulin does not vary more than about 0.5% after at least about 1 month of storage at about 25° C.

44. The preparation of claim 43 wherein the osmolality of a formulation comprising insulin does not vary more than about 0.5% after at least about 6 months of storage at about 25° C.

45. The preparation of claim 44 wherein the osmolality of a formulation comprising insulin does not vary more than about 0.5% after at least about 12 months of storage at about 25° C.

46. The preparation of claim 45 wherein the osmolality of a formulation comprising insulin does not vary more than about 0.5% after at least about 24 months of storage at about 25° C.

47. The preparation of claim 46 wherein the osmolality of a formulation comprising insulin does not vary more than about 0.5% after at least about 36 months of storage at about 25° C.

48. The preparation of claim 1 wherein the insulin content of a formulation comprising insulin is from about 90% to about 115% of the reference product after at least about 1 month of storage at about 5° C.

49. The preparation of claim 48 wherein the insulin content of a formulation comprising insulin is from about 90% to about 115% of the reference product after at least about 6 months of storage at about 5° C.

50. The preparation of claim 49 wherein the insulin content of a formulation comprising insulin is from about 90% to about 115% of the reference product after at least about 12 months of storage at about 5° C.

51. The preparation of claim 50 wherein the insulin content of a formulation comprising insulin is from about 90% to about 115% of the reference product after at least about 24 months of storage at about 5° C.

52. The preparation of claim 51 wherein the insulin content of a formulation comprising insulin is from about 90% to about 115% of the reference product after at least about 36 months of storage at about 5° C.

53. The preparation of claim 1 wherein the insulin content of a formulation comprising insulin is from about 90% to about 115% of the reference product after at least about 1 month of storage at about 25° C.

54. The preparation of claim 53 wherein the insulin content of a formulation comprising insulin is from about 90% to about 115% of the reference product after at least about 6 months of storage at about 25° C.

55. The preparation of claim 54 wherein the insulin content of a formulation comprising insulin is from about 90% to about 115% of the reference product after at least about 12 months of storage at about 25° C.

56. The preparation of claim 55 wherein the insulin content of a formulation comprising insulin is from about 90% to about 115% of the reference product after at least about 24 months of storage at about 25° C.

57. The preparation of claim 56 wherein the insulin content of a formulation comprising insulin is about 90% to about 115% of the reference product after at least about 36 months of storage at about 25° C.

58. The preparation of claim 11 wherein the D₉₀ measurement of the oil droplets in the emulsion of insulin is equal to about 0.4-5 microns after at least about 1 month of storage at about 5° C.

59. The preparation of claim 58 wherein the D₉₀ measurement of the oil droplets in the emulsion of insulin is equal to about 0.4-5 microns after at least about 6 months of storage at about 5° C.

60. The preparation of claim 59 wherein the D₉₀ measurement of the oil droplets in the emulsion of insulin is equal to about 0.4-5 microns after at least about 12 months of storage at about 5° C.

61. The preparation of claim 60 wherein the D₉₀ measurement of the oil droplets in the emulsion of insulin is equal to about 0.4-5 microns after at least about 24 months of storage at about 5° C.

62. The preparation of claim 61 wherein the D₉₀ measurement of the oil droplets in the emulsion of insulin is equal to about 0.4-5 microns after at least about 36 months of storage at about 5° C.

63. The preparation of claim 11 wherein the D₉₀ measurement of the oil droplets in the emulsion of insulin is equal to about 0.4-5 microns after at least about 1 month of storage at about 25° C.

64. The preparation of claim 63 wherein the D₉₀ measurement of the oil droplets in the emulsion of insulin is equal to about 0.4-5 microns after at least about 6 months of storage at about 25° C.

65. The preparation of claim 64 wherein the D₉₀ measurement of the oil droplets in the emulsion of insulin is equal to about 0.4-5 microns after at least about 12 months of storage at about 25° C.

66. The preparation of claim 65 wherein the D₉₀ measurement of the oil droplets in the emulsion of insulin is equal to about 0.4-5 microns after at least about 24 months of storage at about 25° C.

67. The preparation of claim 66 wherein the D₉₀ measurement of the oil droplets in the emulsion of insulin is equal to about 0.4-5 microns after at least about 36 months of storage at about 25° C.
